(19) United States
(12) Patent Application Publication
(10) Pub. No.: US 2010/0139688 A1
(43) Pub. Date: Jun. 10, 2010

(54) DENTAL FLOSS DISPENSER AND METHOD

(76) Inventor: Lisa Marie Musgrave, Indianapolis, IN (US)

Correspondence Address: Brannon & Associates PC, 1 North Pennsylvania Street, Suite 520, Indianapolis, IN, 46204, US

(21) Appl. No.: 12/474,271

(22) Filed: May 28, 2009

Related U.S. Application Data
(60) Provisional application No. 61/135,388, filed on Jul. 21, 2008.

(51) Int. Cl. A61C 15/04; B65H 55/00; A61B 19/02

(52) U.S. Cl. 132/321; 206/388; 206/63.5

(57) ABSTRACT

Dental floss packaging and a method of using same is provided, wherein a plurality of predetermined lengths of dental floss, each sufficient for a single flossing, are disposed within individual sealed compartments in the packaging. Manual force applied to a compartment urges the floss through a cover sheet thereby dispensing the floss for use. The empty compartment provides a visual indication that a particular day's flossing has been completed.

### CROSS REFERENCE TO RELATED APPLICATIONS

This application claims priority to U.S. Provisional Patent Application No. 61/135,388 filed on Jul. 21, 2008.

### FIELD

The claimed technology relates generally to oral healthcare products and more specifically to methods and devices for dispensing dental floss and the monitoring thereof.

### BACKGROUND

Tooth decay and periodontal disease can be caused by bacterial activity resulting from the formation of plaque on the teeth in addition to trapped food particles between teeth and/or between a tooth and the gum. Conventional brushing alone has been found to be inadequate for removing all entrapped food particles and plaque buildup. Daily flossing to remove remaining plaque and entrapped food particles as well as associated bacteria significantly reduces the incidence of dental caries, gingivitis, bad breath, and promotes overall oral health. Additionally, recent research has shown correlations between oral health and other conditions such as prostate cancer, heart disease, diabetes, as well as premature and low birth weight babies.

Yet with all of the benefits associated with flossing, many patients do not floss on a daily basis if at all. According to dental patient surveys, “you need to floss more” is the phrase patients least like to hear from their dentists. One possible reason for failing to floss on a regular basis may be the design of the floss packaging itself. Typically, dental floss is packaged by winding several yards of floss on a spool placed within a plastic housing having a hinged lid. The free end of the floss is allowed to extend upward from the spool so that the desired length of floss can be drawn off of the spool and cut using a cutting blade attached to the housing.

The total length of floss on a spool may vary from brand to brand and typically patients have no way of determining how much floss is remaining in a container, especially if more than one person uses floss from a single container. Patients may use a different length of floss with each use unless they are careful to draw off the same amount at each flossing. The combination of not always being able to tell how much floss is remaining on a spool as well as drawing off varying lengths of floss with each use makes it difficult to tell if a patient has flossed on a particular day. With no ability to verify usage or frequency of flossing, there is little to no accountability for a patient to encourage daily flossing with existing floss dispensers. What is needed is a way for patients to visually verify that a particular day's flossing has been completed; as well as that the proper amount of floss was used; so as to encourage daily flossing and thereby improve both oral and overall health of the patient.

### BRIEF DESCRIPTION OF THE DRAWINGS

FIG. 1 is a top plan view of a dental floss dispenser according to one embodiment of the disclosed technology.

FIG. 2 is a partial cut away side view of the dispenser shown in FIG. 1.

FIG. 3 is a top plan view of a dental floss dispenser according to another embodiment of the disclosed technology.

FIG. 4 is a top plan view of a dental floss dispenser according to still another embodiment of the disclosed technology.

FIG. 5 is a top plan view of a dental floss dispenser according to yet another embodiment of the disclosed technology.

FIG. 6 is a partial cut away side view of the dispenser shown in FIG. 5.

FIG. 7 is a top plan view of a dental floss dispenser according to an embodiment of the disclosed technology.

FIG. 8 is a top plan view of a dental floss dispenser according to a further embodiment of the disclosed technology.

FIG. 9 is a top plan view of a dental floss dispenser according to another embodiment of the disclosed technology.

### DESCRIPTION

For the purposes of promoting an understanding of the principles of the claimed technology and presenting its currently understood best mode of operation, reference will now be made to the embodiments illustrated in the drawings and specific language will be used to describe the same. It will nevertheless be understood that no limitation of the scope of the claimed technology is thereby intended, with such alterations and further modifications in the illustrated device and such further applications of the principles of the claimed technology as illustrated therein being contemplated as would normally occur to one skilled in the art to which the claimed technology relates.

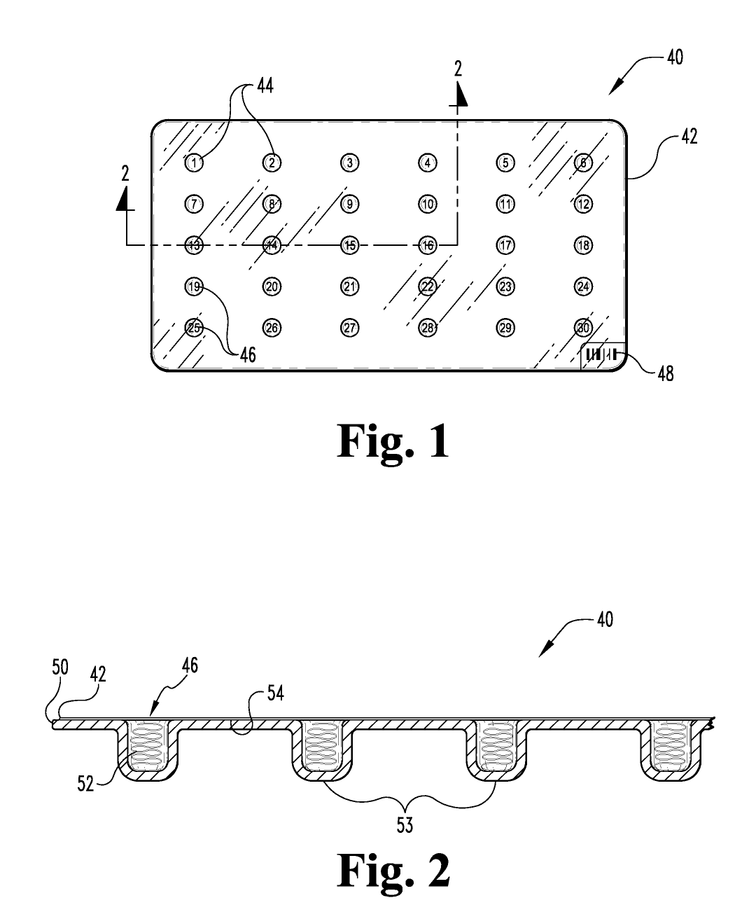

A top plan view of a dental floss dispenser 40 according to one embodiment of the disclosed technology is shown in FIG. 1 while a partial cut away side view of the dispenser 40 shown in FIG. 1 taken along line 2 is shown in FIG. 2. Dental floss dispenser 40 includes a plurality of cavities 53 disposed about the packaging. A coiled length of dental floss 52 having a predetermined length is sealed within each cavity 53. Each individual length of floss has the same predetermined length as every other length of floss, and this predetermined length is selected so as to be suitable for a single use flossing a user's teeth. In one example of the disclosed technology, each premeasured piece of dental floss is of at least eighteen inches in length. In other embodiments, each piece of dental floss is of a greater or lesser length as desired. For example, packaging for adult users may contain one predetermined length of floss while packaging for child users may contain another, shorter, length of dental floss. Floss 52 can be of any desired type, style, diameter, and/or flavor of dental floss or tape.

Dispenser 40 is comprised of a backing sheet 50 about which a plurality of cavities 53 are disposed. In one embodiment, each cavity 53 is formed in the otherwise planar backing sheet 50 of some type of deformable material such as thermoformable plastic sheet material. In another embodiment, backing sheet 50 is a semi-rigid transparent plastic such as cast vinyl. Cavities 53 may be formed by thermoforming a region of a planar plastic material to the desired shape such as by placing the plastic material between a pair of heated dies that are temperature controlled at a temperature sufficient to soften the plastic while pressing the dies together so as to form the desired indentations or “blisters” in the plastic. Such a technique is well known in the packaging art. The cavities or blisters may be of a generally round, hemispherical shape or other shape such as cylindrical, square, rectangular, ovoid, or other suitable shape as desired.

To enclose the floss 52 within cavity 53, a cover sheet 42 is disposed so as to cover each of the plurality of cavities 53. Cover sheet 42 is joined to the backing sheet 50 at one or more suitable locations 54 using a suitable attachment method such as heat, adhesives, or other suitable techniques. In one embodiment, cover sheet 42 is applied in a manner so as to form a hermetic seal with backing sheet 50 to prevent contamination of the floss prior to use. Cover sheet 42 comprises a material so as to allow the floss 52 to be pushed therethrough when sufficient manual pressure is applied to cavity 53. In one example, cover sheet 42 is comprised of a thin metal foil material. In other examples, cover sheet 42 is comprised of plastic, paper, or a composite of two or more suitable types of materials.

The size, configuration, and arrangement of cavities 53 formed in backing sheet 50 are selected in this particular embodiment so that it is possible for a user to apply sufficient manual pressure to an individual cavity 53 so as to deform the cavity and thereby urge the floss 52 contained within that particular cavity through the portion of the cover sheet 44 which seals that particular cavity. Each individual cover sheet portion 44 which seals a particular cavity further includes one or more indicium 46 which indicates a particular day or date. In one example, each cover sheet portion 44 which seals a particular cavity further includes an indicium which is unique from the other cover sheet portion indicia, for example, a number from one to thirty. Such indicia may be printed, stamped, etched, molded, or otherwise generated in or on cover sheet 42 in the appropriate location(s).

In this particular example, dispenser 40 further includes a Radio Frequency Identification (RFID) device 48 capable of recording and transmitting information related to the dispensing of floss from the packaging. RFID device 48 may be active (i.e., having a power source) or passive (i.e., lacking a power source) as desired and may be capable of recording and transmitting such information as how many and how frequently portions of dental floss were dispensed from the dispenser. For example, the RFID device may be able to differentiate between a patient that dispensed floss on a daily basis and one that dispenses all of the floss in a single day (presumably right before returning the dispenser to his dentist). Such a device allows a dentist to track if a patient is actually flossing and with what frequency.

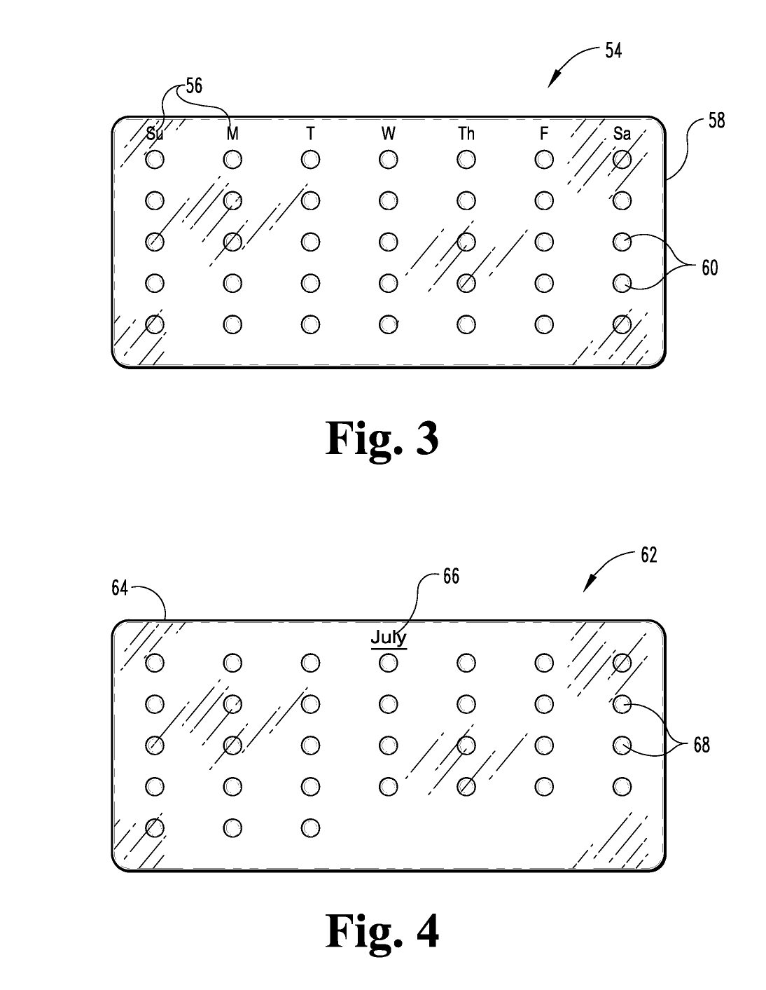

In another example shown in FIG. 3, indicia indicating a particular day of the week (i.e., “Sunday”, “S”, “Su”, or the like as appropriate) may be used. In this particular example, a dental floss package 54 includes a plurality of sealed cavities 60 disposed about a cover sheet 58 in five rows and seven columns for a total of thirty-five sealed cavities containing dental floss. Each column is associated with the indicium 56 of a particular day of the week represented by one of Su (Sunday), M (Monday, T (Tuesday), W (Wednesday), Th (Thursday), F (Friday), or Sa (Saturday). Such a package having thirty-five individual portions of floss would be used over the course of a five week period assuming the patient flossed once per day.

In the example shown in FIG. 4, a dental floss package 62 includes a total of thirty-one sealed cavities 68 containing dental floss disposed about a cover sheet 64. A label 66 indicating “July” is provided. Additionally, each individual sealed cavity 68 may further include a unique number indicium designating a particular day, such as is shown in FIG. 1. In other examples, a package may include thirty, twenty-nine, or twenty-eight individual cavities as appropriate for a particular month.

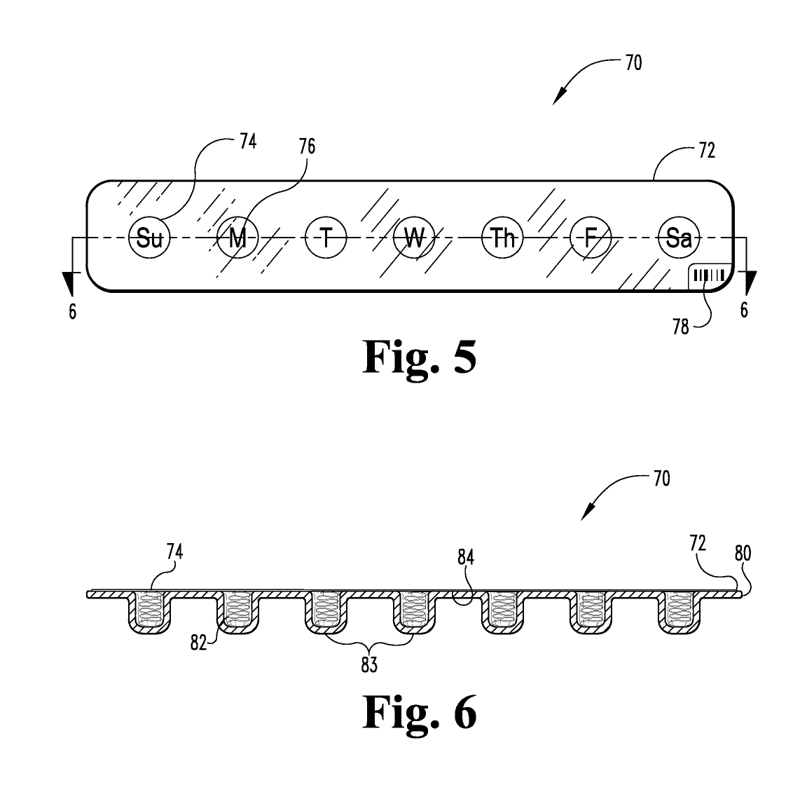

FIG. 5 shows a top plan view of a dental floss dispenser 70 according to yet another embodiment of the disclosed technology, while FIG. 6 is a partial cut away side view of dispenser 70 taken along line 6 in FIG. 5. In this particular embodiment, dental floss dispenser 70 includes a plurality of cavities 83 disposed about the packaging. A coiled length of dental floss 82 having a predetermined length is sealed within each cavity 83. Each individual length of floss has the same predetermined length as every other length of floss, and this predetermined length is selected so as to be suitable for a single use flossing a user's teeth. Floss 82 can be of any desired type, style, diameter, and/or flavor of dental floss or tape.

Dispenser 70 is comprised of a backing sheet 80 about which a plurality of cavities 83 are disposed. In one embodiment, each cavity 83 is formed in the otherwise planar backing sheet 80 of some type of deformable material such as thermoformable plastic sheet material. In another embodiment, backing sheet 80 is a semi-rigid transparent plastic such as cast vinyl. Cavities 83 may be of a generally round, hemispherical shape or other shape such as cylindrical, square, rectangular, ovoid, or other suitable shape as desired.

To enclose the floss 82 within cavity 83, a cover sheet 72 is disposed so as to cover each of the plurality of cavities 83. Cover sheet 72 is joined to the backing sheet 80 at one or more suitable locations 84 using a suitable attachment method such as heat, adhesives, or other suitable techniques. Cover sheet 72 comprises a material so as to allow the floss 82 be pushed therethrough when sufficient manual pressure is applied to cavity 83. In one example, cover sheet 72 is comprised of a thin metal foil material. In other examples, cover sheet 72 is comprised of plastic, paper, or a composite of two or more suitable types of materials.

The size, configuration, and arrangement of cavities 83 formed in backing sheet 70 are selected in this particular embodiment so that it is possible for a user to apply sufficient manual pressure to an individual cavity 83 so as to deform the cavity and thereby urge the floss 82 contained within that particular cavity through the portion of the cover sheet 74 which seals that particular cavity. Each individual cover sheet portion 74 which seals a particular cavity further includes one or more indicium 76 which indicates a particular day or date. In one example, each cover sheet portion 74 which seals a particular cavity further includes an indicium which is unique from the other cover sheet portion indicia, for example, a letter indicating a particular day of the week (i.e., “Sunday”, “S”, “Su”, or the like as appropriate) may be used. Such indicia may be printed, stamped, etched, molded, or otherwise generated in or on cover sheet 72 in the appropriate location(s).

Optionally, dispenser 70 further includes a Radio Frequency Identification (RFID) device 78 capable of recording and transmitting information related to the dispensing of floss from the packaging. RFID device 78 may be active (i.e., having a power source) or passive (i.e., lacking a power source) as desired and may be capable of recording and transmitting such information as how many and how frequently portions of dental floss were dispensed from the dispenser.

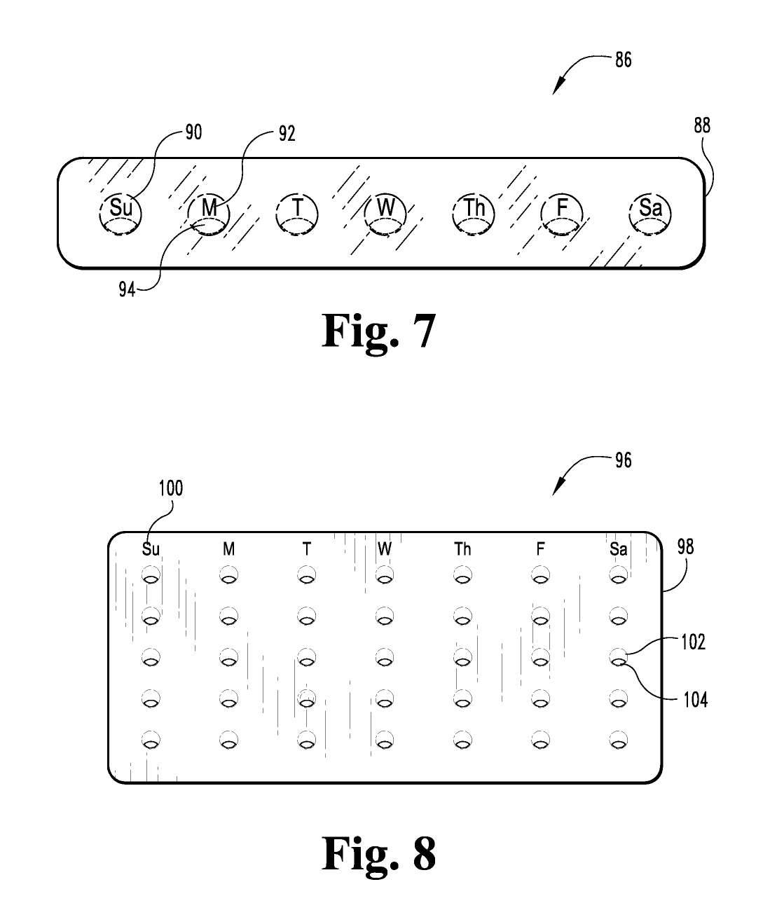

FIG. 7 is a top plan view of a dental floss dispenser 86 according to an embodiment of the disclosed technology. In this particular embodiment, dental floss dispenser 86 includes a plurality of cavities (not shown) disposed about the packaging. The formation and disposition of such cavities are similar to those previously described with respect to the packaging and dispensers shown in FIGS. 1-6. A coiled length of dental floss having a predetermined length is sealed within each cavity in a similar manner to those previously described.

To enclose the floss within each cavity, a cover sheet 88 is disposed so as to cover each of the plurality of cavities. Cover sheet 88 is joined to the backing sheet at one or more suitable locations as previously described. A portion 90 of cover sheet 88 which sealably covers each of the plurality of cavities comprises a perforated tab portion 94. The perforated tab portion 94 is capable of being grasped by a patient such that the manual application of force to the tab 94 will pull or tear away the portion 90 of the cover sheet which seals a particular cavity, thereby dispensing the dental floss contained therein. In one embodiment, the removal of one tab does not remove any other tabs or unseal any other cavities. Cover sheet 88 is comprised of a material such as metal foil, plastic, paper, or a composite material of two or more suitable materials.

Each individual cover sheet portion 90 which seals a particular cavity further includes one or more indicium 92 which indicates a particular day or date. In one example, each cover sheet portion 90 which seals a particular cavity further includes an indicium which is unique from the other cover sheet portion indicia, for example, a letter indicating a particular day of the week (i.e., “Sunday”, “S”, “Su”, or the like as appropriate) may be used. Such indicia may be printed, stamped, etched, molded, or otherwise generated in or on cover sheet 88 in the appropriate location(s).

FIG. 8 is a top plan view of a dental floss dispenser 96 according to another embodiment of the disclosed technology. In this particular embodiment, dental floss dispenser 96 includes a plurality of cavities (not shown) disposed about the packaging. The formation and disposition of such cavities are similar to those previously described with respect to the packaging and dispensers shown in FIGS. 1-6. A coiled length of dental floss having a predetermined length is sealed within each cavity in a similar manner to those previously described.

To enclose the floss within each cavity, a cover sheet 98 is disposed so as to cover each of the plurality of cavities. Cover sheet 98 is joined to the backing sheet at one or more suitable locations as previously described. A portion 102 of cover sheet 98 which sealably covers each of the plurality of cavities comprises a perforated tab portion 104. The perforated tab portion 104 is capable of being grasped by a patient such that the manual application of force to the tab 104 will pull or tear away the portion 102 of the cover sheet which seals a particular cavity, thereby dispensing the dental floss contained therein. In one embodiment, the removal of one tab does not remove any other tabs or unseal any other cavities. Cover sheet 98 is comprised of a material such as metal foil, plastic, paper, or a composite material of two or more suitable materials.

Each individual cover sheet portion 102 which seals a particular cavity further includes one or more indicium which indicates a particular day or date. In one example, cover sheet portions 98 which seals particular cavities are disposed in a series of columns such that each column corresponds to a particular day of the week indicated, for example, by a letter or letters 100 indicating a particular day of the week (i.e., “Sunday”, “S”, “Su”, or the like as appropriate). Such indicia may be printed, stamped, etched, molded, or otherwise generated in or on cover sheet 98 in the appropriate location(s).

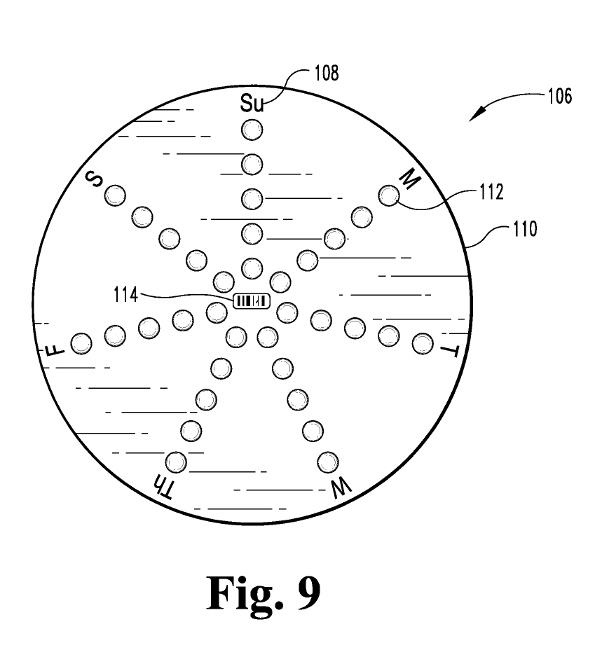

FIG. 9 is a top plan view of a dental floss dispenser according to another embodiment of the disclosed technology. In this particular embodiment, a generally circular shaped dispenser 106 is disclosed. The fabrication and composition of dispenser 106 is generally similar to those dispenser previously disclosed and discussed with respect to FIGS. 1-8. A plurality of sealed cavities 112 are disposed about dispenser 106 and are arranged according to the days of the week as indicated by indicia 108 representing the days Sunday through Saturday as printed on a cover sheet 110. This particular embodiment may further include a Radio Frequency Identification (RFID) device 114 capable of recording and transmitting information related to the dispensing of floss from the packaging.

One or more dispensing devices such as that shown in FIGS. 1-9 may be provided to a patient at a semi-annual cleaning and checkup at a dentist or at some other scheduled time. Between visits to the dentist, the patient is advised to regularly dispense and use the floss from the dispenser. At a follow up visit to the dentist, the patient returns the used dispensers so that the dentist can monitor the patient's progress, or lack thereof, in regularly flossing between dental visits. Dispensers having RFID or other monitoring or tracking devices can then be scanned so as to confirm proper and timely flossing. Dispensers lacking a tracking device may be visually inspected and the level of floss use recorded. This information can then be used by the dentist to counsel the patent so as to improve overall oral health. Additionally, some insurance companies may condition “healthy lifestyle” rate discounts or similar benefits on good habits such as regular flossing. Proof of such habits may be shown using information collected by the dentist through the use of such dispensers and transmission of this information to the insurance company.

While the claimed technology has been illustrated and described in detail in the drawings and foregoing description, the same is to be considered as illustrative and not restrictive in character. It is understood that the embodiments have been shown and described in the foregoing specification in satisfaction of the best mode and enablement requirements. It is understood that one of ordinary skill in the art could readily make a nigh-infinite number of insubstantial changes and modifications to the above-described embodiments and that it would be impractical to attempt to describe all such embodiment variations in the present specification. Accordingly, it is understood that all changes and modifications that come within the spirit of the claimed technology are desired to be protected.

What is claimed is:

1. Packaged dental floss, comprising:
a backing sheet comprising a plurality of cavities having a predetermined size;
a pre-measured length of dental floss disposed within each of said cavities; and
a cover sheet disposed with respect to said cavities to sealably cover said cavities and retain said length of dental floss therein;
wherein said backing sheet comprises a material constructed to deform in response to manually applied pressure to said cavity and urge said length of dental floss through said cover sheet so as to dispense said dental floss;
wherein the portion of said cover sheet covering each cavity includes a unique day indicium.

2. The packaged dental floss of claim 1, wherein said cover sheet further includes a month indicium.

3. The packaged dental floss of claim 2, wherein said backing sheet comprises a number of cavities corresponding to the number of days in the indicated month.

4. The packaged dental floss of claim 1, wherein said cover sheet comprises a metal foil.

5. The packaged dental floss of claim 1, wherein said backing sheet comprises transparent plastic.

6. The packaged dental floss of claim 1, wherein said pre-measured length of dental floss is of a length suitable for a single use to floss an adult user's teeth.

7. The packaged dental floss of claim 1, wherein said pre-measured length of dental floss is of a length suitable for a single use to floss a child user's teeth.

8. The packaged dental floss of claim 1, wherein said backing sheet comprises seven cavities.

9. The packaged dental floss of claim 1, further comprising a device capable of recording each act of dispensing.

10. The packaged dental floss of claim 9, wherein said device is an RFID device.

11. The packaged dental floss of claim 1, wherein dispensing said dental floss through said cover sheet defaces said day indicium.

12. A dental floss dispenser, comprising:
a backing sheet defining a plurality of cavities;
a plurality of pre-measured lengths of dental floss, one said length of dental floss being disposed in each of said cavities; and
a cover sheet having a plurality of perforated tabs disposed to sealably cover said cavities and retain said length of dental floss therein;
wherein said perforated tabs are configured and arranged such that a tab sealably covers one cavity;
wherein said perforated tabs comprise a material constructed to tear away in response to manual urging to a respective tab to reveal the corresponding cavity and dispense said length of dental floss contained therein.

13. The packaged dental floss of claim 12, wherein each perforated tab further includes a unique day indicium.

14. The packaged dental floss of claim 12, wherein said cover sheet further includes a month indicium.

15. The packaged dental floss of claim 14, wherein said backing sheet comprises a number of cavities corresponding to the number of days in the indicated month.

16. The packaged dental floss of claim 12, further comprising an RFID device capable of recording each act of dispensing.

17. A method of dispensing dental floss and reporting the use thereof, comprising the steps of:
providing a patient with at least one dental floss packaging on a first occasion, said packaging having plurality of cavities, a pre-measured length of dental floss sealed within each of said cavities by a cover sheet comprising a material such that manually applied pressure to said cavity urges said dental floss through said cover sheet so as to dispense said dental floss;
collecting said at least one packaging from said patient at a later occasion;
noting the number of lengths of dental floss dispensed from said at least one packaging; and
recording the total number of acts of dispensing said dental floss by the patient.

18. The method of claim 17, wherein said packaged dental floss further comprises an RFID device capable of recording each act of dispensing.

19. The method of claim 17, further comprising the step of providing said total number of acts of dispensing said dental floss by the patient to an insurance company.

20. The method of claim 17, wherein at least one dental floss packaging further includes a unique day indicium printed on said cover sheet for each length of dental floss.
